(19) United States
(12) Patent Application Publication
(10) Pub. No.: US 2019/0311809 A1
(43) Pub. Date: Oct. 10, 2019

(54) PATIENT STATUS MONITOR AND METHOD OF MONITORING PATIENT STATUS

(71) Applicant: Oxford University Innovation Limited, Oxford (GB)

(72) Inventors: Duncan YOUNG, Oxford (Oxfordshire) (GB); Peter WATKINSON, Oxford (Oxfordshire) (GB); Lionel TARASSENKO, Oxford (Oxfordshire) (GB)

(21) Appl. No.: 16/463,404

(22) PCT Filed: Oct. 31, 2017
(86) PCT No.: PCT/GB2017/053271

§ 371 Date: May 23, 2019

(30) Foreign Application Priority Data
Nov. 24, 2016 (GB) 1619902.8

(51) Int. Cl. G16H 50/30; A61B 5/0205; G16H 10/60; A61B 5/00

(52) U.S. Cl. CPC G16H 50/30; A61B 5/02055; G16H 50/20; A61B 5/7275; A61B 5/742; G16H 10/60

(57) ABSTRACT

A patient status monitor for providing an estimate of the risk of an adverse health event such as death or intensive care readmission based on a first risk estimate using patient data collected over a first time period, such as a stay in an intensive care unit, and a second risk estimate based on current vitals signs measurements. The first and second risk estimates are based on different models, the first being a static logistic regression on data selected from electronic patient records, and the second being a novelty detection algorithm based on a a training data set of vital signs measurements representing normality. The two risk estimates are combined in a weighted combination, with the first risk estimate having a weight which decreases with time from the end of the first time period.

The present invention relates to a patient status monitor and method of monitoring patient status to provide an estimate of the risk of a future decline in the patient's health.

A variety of systems have been proposed for monitoring patient's vital signs and predicting adverse events such as cardiac arrests or unplanned admission to a higher care area (such as an intensive care unit) or for giving a single visual indication, such as a score, of the current status of the patient. For example, simple early warning score (EWS) systems (also called “Track and Trigger” systems) have been developed since 1997 such as described in “An early warning scoring system for detecting developing critical illness” by R M Morgan, F Williams and M M Wright, Clin. Intensive Care, 1997. 8 (2) p.100, and “Review and Performance Evaluation of Aggregate Weighted “Track and Trigger Systems” by G B Smith et al., Resuscitation, 2008. 77 (2): pp170-179. These systems usually specify a number of bands covering all the possible values of each regularly recorded vital sign. They then either mandate an action when one or more of the vital signs enters the most extreme bands (multiple parameter systems), or assign integer scores to the bands which increase as the bands become less normal. The sum of the scores for all the vital signs are then compared to threshold values to determine actions (aggregate weighted systems). These EWS systems evolved from paper-based vital signs charts and have been implemented on purpose-designed paper charts although electronic systems are now commonplace. Such EWS systems use vital signs in part because of their historical link to vital signs observation charts, and partly because vital signs are the most frequently-recorded measures of a patient's condition.

More recently models have been developed which process vital signs measurements using novelty detection, or one-class classification, which involves the construction of a multivariate, multimodal model of normality using a development data set containing vital signs that are normally seen in the target patient population. By comparing new sets of vital signs measurements to the model, a probability is generated that the new vital signs data can be classified as “normal” with respect to the development data set. One example of such a system is the Visensia® safety index developed by OBS Medical and described in U.S. Pat. No. 7,031,857.

While such systems have been beneficial in successfully predicting deterioration in some patients' health before the deterioration becomes critical, when assessing a patient's condition clinical staff typically factor in much more information than only vital signs. For example, demographic information such as the patient's age and background, information on what treatments the patient has previously received and the response, information from laboratory tests of patient tissue or fluid. The integration of such factors into a risk estimate is, however, typically a matter of experience.

The present invention provides a patient status monitor and method of monitoring a patient which allows the combination of data collected during a first predetermined time period with current vital signs measurements in order to provide a combined estimate of the risk of the patient's health declining. In one embodiment this is achieved by determining, at the end of a first time period, a first risk estimate based on patient data collected up to the end of that first time period, separately determining a second risk estimate based on a vital signs measurement in a second time period, and forming a weighted combination of the first and second risk estimates, with the weight of the first risk estimate decreasing with time since the end of the first time period. The weighted combination is displayed as the overall, combined risk estimate. Thus the first risk estimate is based on a set of data collected up to the end of the first time period, whereas the second risk estimate is a dynamic risk estimate based on a current vital signs measurement. As the first risk estimate becomes older with respect to the current measurement time, its influence on the combined risk estimate is decreased. The first risk estimate is a static risk estimate based on the data collected up to the end of the first time period, whereas the second risk estimate is a dynamic risk estimate which is updated with new vital signs measurements.

By separating out the static risk estimate from the dynamic risk estimate it is possible to use different techniques for determining the risk estimates from the respective data sets. Further, it is possible to base the second risk estimate on vital signs data over a time period which is different from the first time period over which the data for the first, static, risk estimate was collected. The first risk estimate may be based on dense data, i.e. many observations over a short time period, whereas the second risk estimate may be based on sparse data—such as just the vital signs readings taken more infrequently than the first set, and over a longer time period.

The invention is particularly useful for patients who spent a first period of time critically ill in an intensive care unit, when their vital signs could typically be varying wildly, and then spend a second period of time in a general ward. Although such patients have been discharged from an intensive care unit, they do not all recover uneventfully, and some die or have to be readmitted to intensive care. Thus the invention can provide an estimate of the risk of an adverse health event such as a death or readmission to intensive care based on (i) a static risk estimate determined by patient data collected while in intensive care and (ii) a second, dynamic, risk estimate based on vital signs measurements made (usually periodically) on the general ward.

The risk of a future decline in patient health may be the risk of an adverse health event occurring, for example within a predetermined period of time, such as death, or risk of re-admission to a higher care facility such as an intensive care unit.

The patient data for the first risk estimate preferably includes one or more of: physiological variables recorded during the first time period, patient demographics, details of treatments received during the first time period, response to those treatments, and results of in vitro tissue or fluid analysis (laboratory tests). The patient data for the first risk estimate are observations of those factors which are found by analysis of training data to have high correlation with future decline in patient's health, more preferably a specific event such as death or readmission to a higher care facility. A predetermined number of the most significant factors, for example at least 20, more preferably at least 30, more preferably between 40 and 50 of the most significant factors from those monitored during the first time period, e.g. the stay in an intensive care unit (ICU).

The first risk estimate may be determined by a number of models that estimate risk, one of which is logistic regression using a logistic regression model developed on a training data set comprising recorded patient data for many patients together with each patient's subsequent health, such as whether they suffered a decline in health or an adverse health event such as death or readmission to a higher care unit.

The first and second risk estimates may be initially combined with equal weight, or the second risk estimate—based on vital signs—may start with a different, e.g. higher or lower weight in the combination than the first risk estimate. The weight of the first risk estimate in the combined risk estimate may be set to decay by less than one percent per hour since the end of the first time period, for example from 0.1%-0.9% per hour, more preferably from 0.3% to 0.7% per hour, yet more preferably from 0.5 to 0.6% per hour.

Preferably the vital signs measured to compute the second risk estimate comprise at least the patient's heart rate, respiratory rate, blood pressure, body temperature and arterial oxygen saturation. These vital signs may not all be measured simultaneously or at the same rate and so the second risk estimate may be recalculated every time one of the vital signs being measured is updated. The second risk estimate may be determined by using novelty detection by comparing the current vital signs to a model of normality to obtain a probability that the current vital signs are normal. The model of normality may be a multivariate, multimodal model of normality developed on a training data set of vital signs observations for many patients regarded as normal.

Preferably a new weighted combination of the two risk estimates is formed every time a new vital signs measurement is received and a new second risk estimate determined.

The combined risk estimate may be displayed as a number and significant increases in risk or a combined risk above a threshold may trigger alerts to clinical staff.

The system is well adapted to monitoring plural patients in which case the individual patient's risks are displayed, optionally with an indication of the trend of that risk estimate. The patients may be ranked by overall risk. The status monitor is also adapted to display to the clinician the main factors influencing the current risk estimate, preferably separating risk associated with the patient data collected over the first time period and the current vital signs data. This allows the clinician to confirm for themselves the risk estimate made by the status monitor. By displaying the risk estimates for plural patients and allowing them to be ordered by risk, the clinician can prioritise their attention to those patients with high risk.

The invention may be embodied by using a general purpose computer programmed to receive the patient data and vital signs measurements and to calculate the risk estimates, combine them and display the results, and so the invention extends to a computer program for controlling such a computer to execute the invention. Alternatively the invention may be embodied as, or as part of, a dedicated patient monitor, which receives the patient data and vital signs measurements, or optionally makes the vital signs measurements, and which includes a data processor adapted to determine and display the risk estimate.

The invention will be further described by way of example with reference to the accompanying drawings in which:—

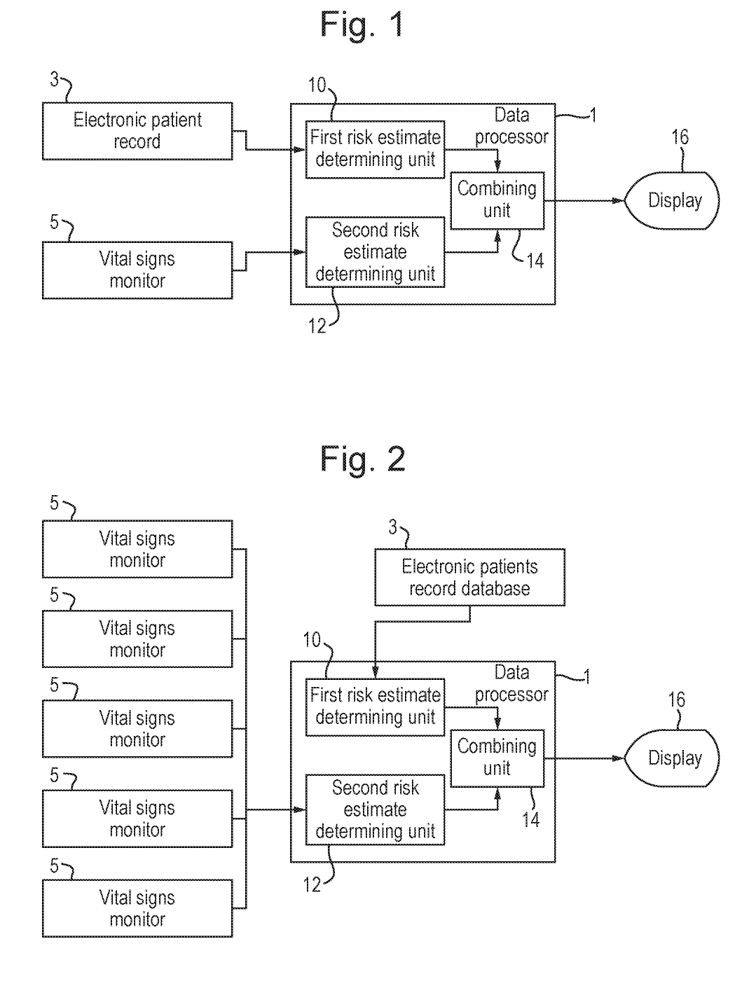

FIG. 1 schematically illustrates a patient's status monitor in accordance with an embodiment of the invention;

FIG. 2 schematically illustrates a status monitor monitoring plural patients;

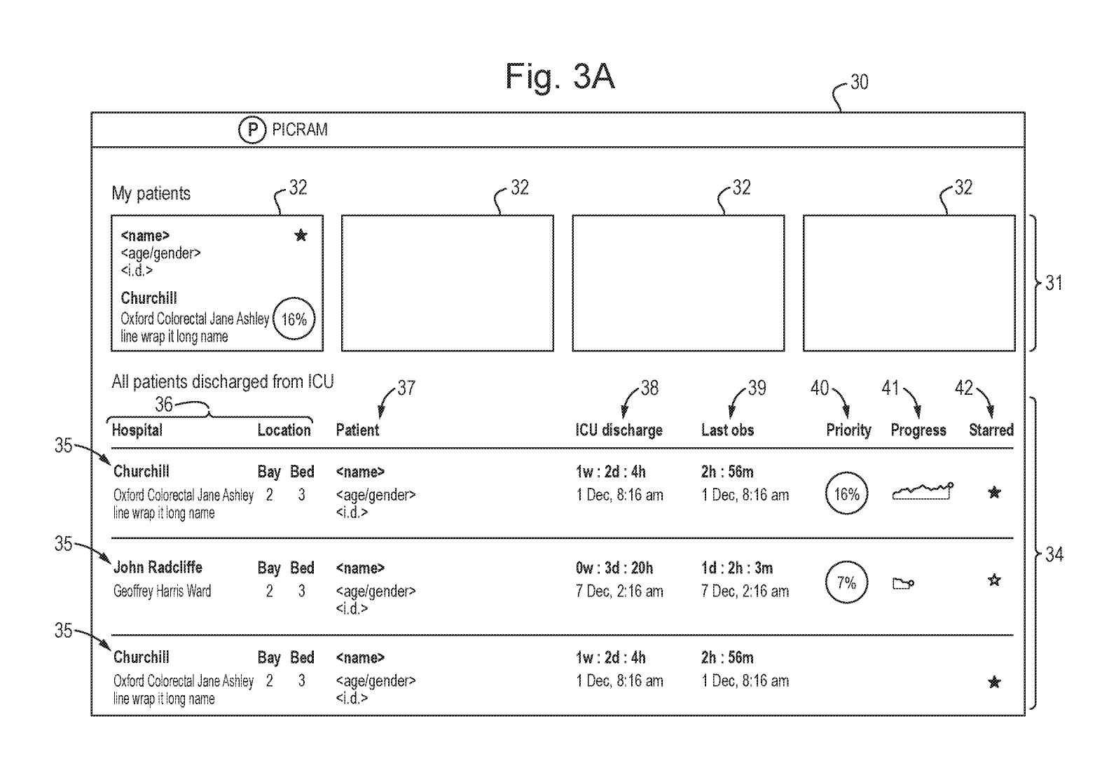

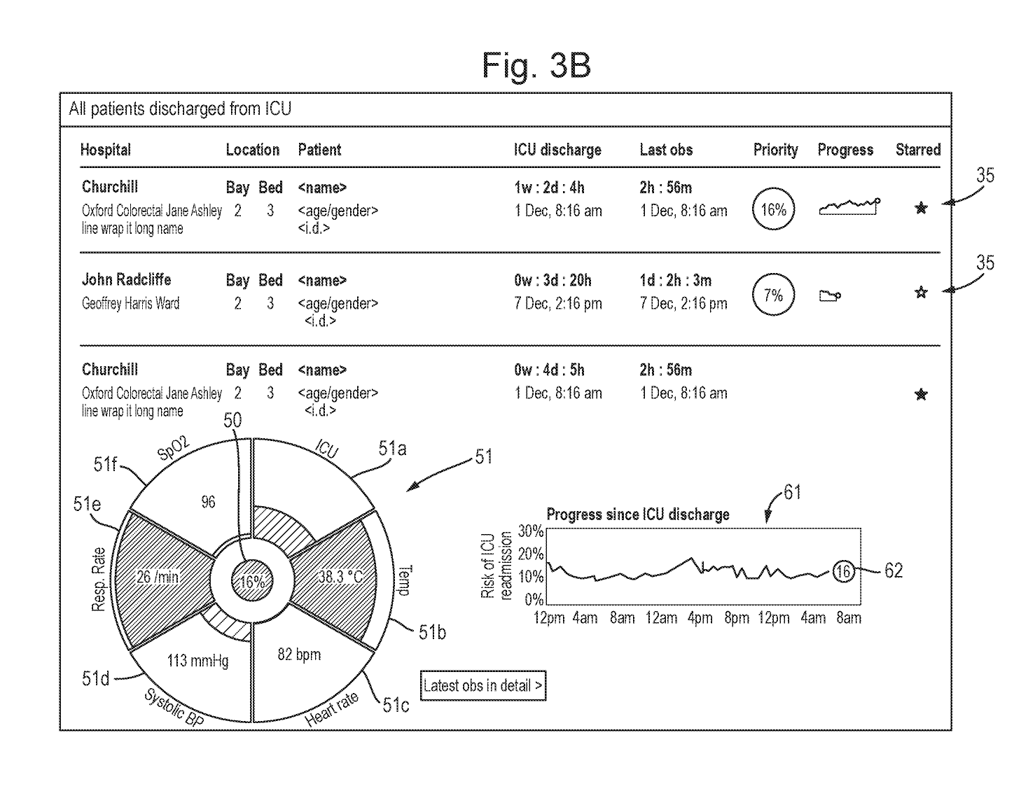

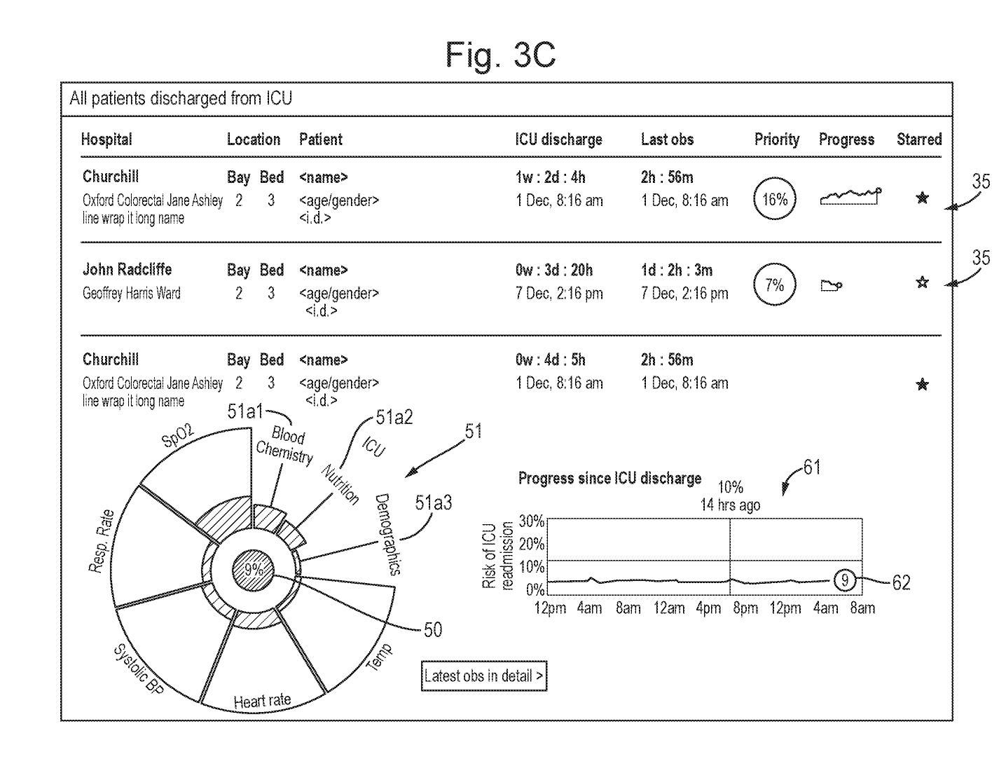

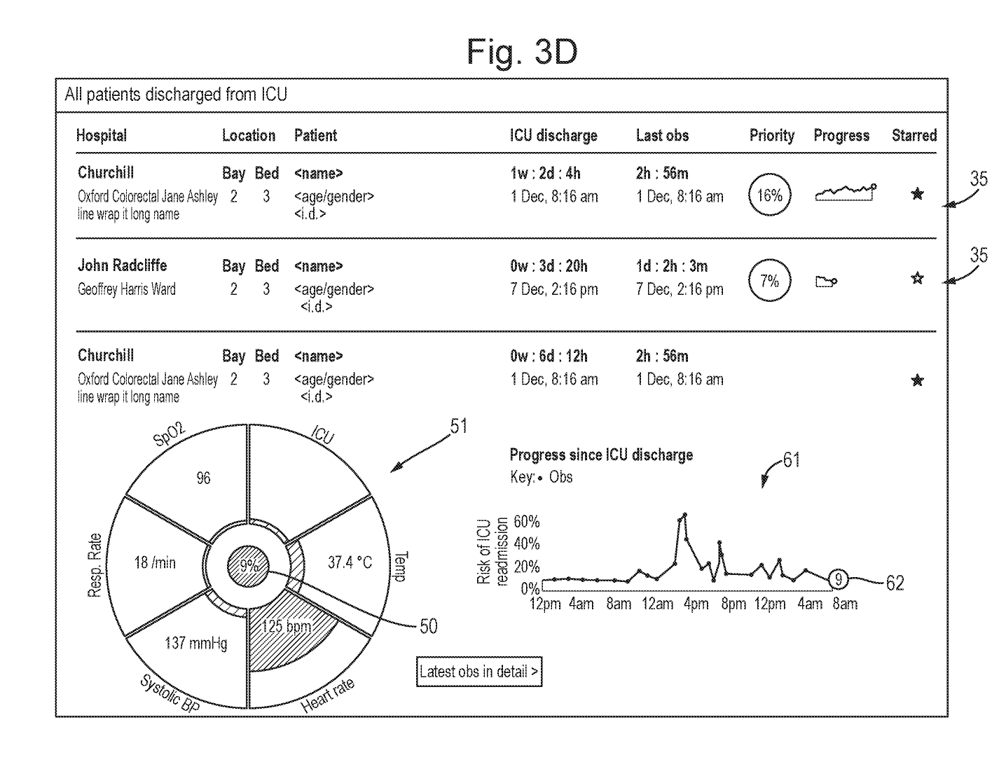

FIGS. 3A to D illustrate schematically example displays from one embodiment of the invention monitoring multiple patients.

FIG. 1 schematically illustrates a patient status monitor in accordance with a first embodiment of the invention. It comprises a data processor 1, which can be a suitably programmed general purpose computer or a data processor of a dedicated patient monitoring system, which is adapted to receive patient data collected over a first time period, in this case from an electronic patient record 3. Although in this embodiment the electronic supply of patient data from an electronic patient record is envisaged, patient data collected over the first time period may be manually entered into the data processor 1, e.g. by means of a keyboard, if an electronic patient record is not available.

The data processor 1 is also adapted to receive vital signs measurements from a vital signs monitor 5. This provides vital signs such as a heart rate, respiration rate, blood pressure, arterial blood saturation and temperature. Although the vital signs monitor 5 is illustrated as a single unit, in an alternative embodiment separate monitors for each of the vital signs may supply their measurements to the data processor 1. The vital signs monitors may be part of a dedicated patient monitoring system, or may be separate commercially-available monitors, or may be a manual-entry computer based system such as VitalPac or SEND.

The data processor 1 includes a first risk estimate determining unit 10 and a second risk estimate determining unit 12. These are preferably embodied as software for controlling the data processor to process the incoming data and determine the risk estimates as explained below.

The first risk estimate determining unit 10 receives the patient data collected over the first time period and determines a first risk estimate, which is output to a combining unit 14. In this embodiment the first risk estimate determining unit 10 determines the risk estimate from the patient data by using logistic regression. In this embodiment a subset of 42 of the individual data items recorded daily for patients in an intensive care unit were used, as listed in Table 1 below. The variable name and collection rule are shown.

Although all 42 data items are used in this embodiment, the invention contemplates the use of a different number or different observations. The data items to be used are those which are found in a training set of data to be factors showing highest correlation with the outcome—in this case death or readmission to ICU. Thus the invention contemplates using a subset of the items listed below—for example the top ten, twenty or thirty most significant.

| Variable | Collection rule |
|---|---|
| SodiumLast_EXTREMEFLAG | The last plasma sodium concentration before ICU discharge |
| | was in the extremes of the development set range |
| ParenteralFeedingStayMax | If parenteral feeding was used at any point during ICU stay |
| EnteralFeedingStayMax | If enteral feeding was used at any point during ICU stay |
| Age | Age at ICU admission |
| UreaLast | Last plasma urea concentration before ICU discharge |
| RRFirst24Min_EXTREMEFLAG | The lowest respiratory rate in the first 24 hours of ICU |
| | admission was in the extremes of the development set range |
| UrineOutputLast24Sum_EXTREMEFLAG | The urine output in the last 24 hours of ICU admission was |
| | in the extremes of the development set range |
| WCCLast72Min_EXTREMEFLAG | The lowest white cell count in the last 72 hours of ICU |
| | admission was in the extremes of the development set range |
| HRLast | The last ventricular heart rate before ICU discharge |
| DysrhythmiaLast24Max | Any dysrhythmia in the last 24 hours of ICU admission |
| SodiumLast | The last plasma sodium concentration before ICU discharge |
| CRPLastHIGHFLAG | The last CRP before ICU discharge was >156 mg/l |
| PreICULOS | The time between acute hospital and ICU admission in days |
| RRLast72Max | The highest respiratory rate in the last 72 hours of ICU |
| | admission |
| HRFirst24Max | The highest ventricular heart rate in the first 24 hours of |
| | ICU admission was in the extremes of the development set range |
| BilirubinLast24Max | The highest plasma bilirubin in the last 24 hours of ICU |
| | admission |
| WCCLast_EXTREMEFLAG | The last white cell count before ICU discharge was in the |
| | extremes of the development set range |
| LactateBGLast | The last plasma lactate before ICU discharge |
| InsulinLast24Max | If an insulin infusion was used in the last 72 hours of ICU |
| | admission |
| RRLast72Min | The lowest respiratory rate in the last 72 hours of ICU |
| | admission |
| NumVasoactiveDrugsFirst24Max | The maximum number of vasoactive drugs used in the first 24 |
| | hours of ICU admission |
| RRFirst24Min | The lowest respiratory rate in the first 24 hours of ICU |
| | admission |
| AaDO2Last | The last alveolar-arterial oxygen tension gradient before ICU |
| | discharge |
| CRPFirst24Max | The highest CRP in the first 24 hours of ICU admission |
| RRLast | The last respiratory rate before ICU discharge |
| SodiumFirst24Max_EXTREMEFLAG | The highest plasma sodium concentration during the fist 24 hours of ICU stay was in |
| | the extremes of the development set range |
| BilirubinLast | The last plasma bilirubin concentration before ICU discharge |
| HRLast72Min | The lowest ventricular heart rate in the last 72 hours of ICU admission |
| SodiumFirst24Min_EXTREMEFLAG | The lowest plasma sodium concentration during the fist 24 hours of ICU stay was in |
| | the extremes of the development set range |
| PotassiumFirst24Min | The lowest plasma potassium in the first 24 hours of ICU admission |
| SpO2Last72Min | The lowest arterial oxygen saturation recorded using pulse oximetry in the last 72 |
| | hours of ICU admission |
| GCSFirst24Min | The lowest Glasgow Coma Score in the first 24 hours of ICU admission |
| TempLast72Max | The highest temperature in the last 72 hours of ICU admission |
| TempLast72Min | The lowest temperature in the last 72 hours of ICU admission |
| WeightFirst24Max | The highest weight in the first 24 hours of ICU admission |
| TempLast | The last temperature recorded before ICU discharge |
| PaFiLast24Min | The lowest PaO₂/FiO₂ ratio in the last 24 hours of ICU admission |
| VentilatedLast24Max | The use of artifical ventilation in the last 24 hours of ICU admission |
| ABPMeanLast | The last mean arterial blood pressure before ICU discharge |
| PaFiLast | The last PaO₂/FiO₂ ratio before ICU discharge |
| GCSTotalLast | The last Glasgow Coma Score before ICU discharge |
| UrineOutputLast24Sum | The total urine output in the last 24 hours of ICU admission |
| AlbuminLast | The last plasma abumin concentration before ICU discharge |

In this set of data, treatments are converted to dichotomous variables by recording if they were ever used, or if they were in use at discharge from the intensive care unit.

Some variables such as serum, electrolytes or body temperature show a non-linear relationship with adverse outcomes, with extreme high or low values associated with worse outcomes compared with mid-range normal values. For these variables, additional dichotomous variables were generated to indicate if the value was in the most extreme upper or lower 5% of the observed range. A different percentage of the range may be used if desired. CRP (c-reactive protein) values reported as a continuous variable up to 156 mgl⁻¹ and simply as “over 156 mgl⁻¹” thereafter and so were converted to a dichotomous variable. All continuous variables were converted to Z scores (zero mean, unity variance).

To develop the static model which the first risk estimate determination unit 10 uses to calculate risk, data from two general adult intensive care units using 7224 patient records was input to a standard LASSO-penalised logistic regression within a four-fold cross-validation procedure with an outcome variable of combined in-hospital death and ICU readmission. This produces a static, logistic regression model, which can be used to calculate a first risk estimate on a new set of patient data.

Alternative ways of calculating the first risk estimate would be to use standard non-linear methods such as support vector machines, random forests, or neural networks which are trained on the same set of data and then used to provide a risk estimate based on the input patient data.

The second risk estimate determining unit 12 in this embodiment utilises the novelty detection, or one-class classification technique which involves the construction of a multivariate, multimodal model of normality using a development data set containing vital signs of a patient or patients whose condition has been classed as normal. The model of normality may be based on the patient's own vital signs recorded over a period in which they are assessed by clinicians as normal, or may be vital signs measurements for other patients—again who have been assessed as normal. A method for developing such a model has been described in Tarassenko, L., Clifton, D. A., Pinsky, M. R., Hravnak, M. T., Woods, J. R. and Watkinson, P. J., 2011, “Centile-based early warning scores derived from statistical distributions of vital signs.” Resuscitation, 82(8), pp. 1013-1018.

In this embodiment the vital signs used for model development were the vital signs for over 400 patients of intensive care units, in particular the vital signs recorded for the 24 hours prior to discharge home or at 14 days post-intensive care unit discharge if the patient had not been discharged, died or readmitted. The model allows the generation of a probability that a new set of vital signs data can be classified as normal with respect to the development data set. The techniques for developing the model are fully described in Pimentel, M. A., Clifton, D. A., Clifton, L., Watkinson, P. J. and Tarassenko, L., 2013. Modelling physiological deterioration in post-operative patient vital-sign data. Medical & biological engineering & computing, 51(8), pp. 869-877., which is incorporated herein by reference. The estimate is updated every time one or more vital signs are recorded, thus outputting a dynamic risk estimate as a continuous variable.

The risk estimates from the first risk estimate determining unit 10 and the second risk estimate determining unit 12 are combined by a combining unit 14, again preferably embodied as software for controlling the data processor. In this embodiment the combining unit 14 adds them to produce a single risk estimate which is updated every time a vital sign was recorded, producing a new second risk estimate. The relative importance in the combined risk estimate of the first risk estimate is decreased with time by multiplying the first risk estimate by a time-dependent coefficient w(t):

combined risk estimate=second risk estimate+{first risk estimate)×w(t)}

In this embodiment the coefficient w(t) is set to (1−W times the number of hours since intensive care unit discharge). W may be set to be in the range 0.03 to 0.07, more preferably 0.04 to 0.06, for example about 0.05. Where negative coefficients result, they are rescaled to zero.

In a typical use environment the first risk estimate is based on patient data collected during a patient's stay in an intensive care unit, and is generated at the point of discharge from the intensive care unit into a lower care facility such as a general ward. From that point onwards the first risk estimate, which does not change and is thus “static”, is combined, with decreasing weight with time, with a second risk estimate obtained from the vital signs monitor 5 and based on current vital signs, and thus “dynamic”.

The combined risk estimate determined by the combining unit 14 is displayed on a display 16. It may be displayed in the form of a number, and optionally significant increases in the risk estimate, or high risk estimates may also trigger an alert in the form of a visual or audible alarm locally or remotely using electronic communication. The risk estimates and/or alerts or alarms may also be transmitted to a clinician's pager or personal equipment such as a smartphone or tablet device. The display 16 preferably indicates the trend of the risk estimate for the patient.

The clinician will likely want to see details of why the system thinks the patient is at risk. Clicking on the display 16 will bring up a second screen which gives the top (e.g. five) reasons why risk estimate is high, divided up into risk associated with events on the intensive care unit (and thus contributing to the first risk estimate), and the subsequent vital signs (contributing to the second risk estimate). The clinician is able to click through to screens summarising the trends in the individual vital signs.

FIG. 2 schematically illustrates the patient status monitor monitoring plural patients and providing a single display. In this case each patient will have a vital signs monitor 5 (or set of individual vital signs sensors) which supply the vital signs measurement to the data processor 1, while each patient's data collected from the preceding, first, time period may be supplied from a single electronic patient record database 3.

The display 16 displays all the patients who have been discharged from the intensive care unit and are in the hospital. The display 16 also shows their location (ward and bed number) and basic details (name, age, time in hospital etc). Each patient has a risk estimate indication (as an infographic), and preferably an indication of trend. The clinician can rank the patients by risk index (default), or sort by location. The clinician can then construct a visit list, prioritising those patients with high scores (risk indices) first. This would allow the hospital to use a scarce resource (the follow-up nurse) to treat the patients most likely to benefit (those with high risk indices).

FIGS. 3A to D illustrate schematically example displays from one embodiment of the invention monitoring multiple patients.

FIG. 3A illustrates a summary screen 30 in which a clinician can choose to list specific patients in a “My Patients” upper pane 31 of the screen, with each patient having there a summary window 32 with their basic identity, location and current risk estimate 33 displayed numerically and colour coded for different levels of concern. In a lower pane 34 of the screen all patients discharged from ICU are listed—each having a summary entry 35 with their location 36, identity 37, date of and elapsed time since ICU discharge 38, date of and elapsed time since last observation 39, their risk estimate 40 displayed numerically and colour coded (the entries may be listed in order of this value), a short time plot 41 of the evolution of the risk estimate, and a selection indicator 42. Both the upper and lower panes 31, 34 are configurable as desired and the order of listing is selectable on the different data items.

Selecting an individual patient's summary entry 35, e.g. by clicking on it, expands it to show more detail about that patient as illustrated in FIGS. 3B to D.

In each of FIGS. 3B to D a different patient's record is shown. In each case the risk estimate 50 is displayed numerically and colour coded in the centre of a pie chart display 51 in the bottom-left pane which has different segments 51a to 51f for the different contributing parameters to the risk estimate—in this case the ICU-discharge calculated first risk estimate in one segment 51a, and the five vital signs contributing to the second risk estimate: temperature, heart rate, blood pressure, respiration rate and blood oxygen saturation (SpO2). Each segment 51a-f is radially coloured out to a radius which represents its contribution to the risk estimate and includes the most recent observation numerically displayed. The segment 51a corresponding to the first risk estimate can be further subdivided in response to selection by the user (e.g. double clicking on it) to show the top contributors to that first risk estimate, as illustrated in 51a1 to 51a3 of FIG. 3C. In the bottom-right pane a time plot 61 of the risk estimates since ICU discharge is displayed, again together with the current risk estimate number 62, so that the progress since ICU discharge can be seen. A comparison of the pie chart displays of FIGS. 3 B, C and D show how they allow the clinician to see at a glance what is contributing to the particular level of risk estimated. In FIG. 3B the respiration rate and temperature are the high contributors, whereas in FIG. 3C it is the SpO2 and ICU-discharge first estimate, and in FIG. 3D the heart rate.

Of course the invention is not limited to this particular style of display, though it offers complex information in an intuitive and easily understandable way.

1. A patient status monitor for providing an estimate of the risk of a future decline in patient health, comprising:
a data processor adapted to: receive patient data collected over a first time period and determine a first risk estimate, the first risk estimate being the risk at the end of that first time period of a future decline in patient health;
receive during a second time period following the first time period measurements of plural different vital signs of the patient and determining from them a second risk estimate; form a weighted combination of the first and second risk estimates with the weight of the first risk estimate in the weighted combination decreasing with time since the end of the first time period; and
a display adapted to display said weighted combination as said estimate of the risk of a future decline in patient health.

2. A patient status monitor according to claim 1 wherein the second risk estimate is updated, and a new weighted combination with the first risk estimate is formed, every time a new vital signs measurement is received.

3. A patient status monitor according to claim 1 wherein the risk of a future decline in patient health is the risk of an adverse health event occurring within a predetermined period of time in the future.

4. A patient status monitor according to claim 3 wherein the adverse health event is death or re-admission to intensive care unit.

5. A patient status monitor according to claim 1, wherein the patient data comprises physiological variables recorded during said first time period, patient demographic data, data on medical treatment received during said first time period, and results of patient tissue or fluid analysis.

6. A patient status monitor according to claim 5 wherein the patient data comprises a predetermined number of data items selected from: physiological variables recorded during said first time period, patient demographic data, data on medical treatment received during said first time period, and results of patient tissue or fluid analysis.

7. A patient status monitor according to claim 6 wherein the predetermined number of data items is selected by reference to a training data set as those having the highest correlation with the adverse health event.

8. A patient status monitor according to claim 1 wherein the first risk estimate is determined by logistic regression.

9. A patient status monitor according to claim 1 wherein the vital signs measurements comprise measurements of the heart rate, respiratory rate, blood pressure, body temperature and arterial oxygen saturation of the patient.

10. A patient status monitor according to claim 1 wherein the second risk estimate is determined by novelty detection by comparing the vital signs measurements to a multivariate, multimodal model of normality and obtaining a probability that the current vital signs are normal.

11. A patient status monitor according to claim 1 wherein the display is adapted to display a plurality of factors most significantly influencing the combined risk estimate.

12. A method of monitoring patient status to provide an estimate of the risk of a future decline in patient health, comprising the steps of:
receiving patient data collected over a first time period and determining a first risk estimate, the first risk estimate being the risk at the end of that first time period of a future decline in patient health; receiving during a second time period following the first time period measurements of plural different vital signs of the patient and determining from them a second risk estimate; forming a weighted combination of the first and second risk estimates with the weight of the first risk estimate in the weighted combination decreasing with time since the end of the first time period; and
displaying said weighted combination as said estimate of the risk of a future decline in patient health.

13. A method of monitoring patient status according to claim 12 wherein the risk of a future decline in patient health is the risk of an adverse health event occurring within a predetermined period of time in the future.

14. A method of monitoring patient status according to claim 13 wherein the adverse health event is death or re-admission to intensive care unit.

15. A method of monitoring patient status according to claim 12, wherein the patient data comprises physiological variables recorded during said first time period, patient demographic data, data on medical treatment received during said first time period, and results of patient tissue or fluid analysis.

16. A method of monitoring patient status according to claim 15 wherein the patient data comprises a predetermined number of data items selected from: physiological variables recorded during said first time period, patient demographic data, data on medical treatment received during said first time period, and results of patient tissue or fluid analysis.

17. A method of monitoring patient status according to claim 16 wherein the predetermined number of data items is selected by reference to a training data set as those having the highest correlation with the adverse health event.

18. A method of monitoring patient status according to claim 12, wherein the first risk estimate is determined by logistic regression.

19. A method of monitoring patient status according to claim 12, wherein the vital signs measurements comprise measurements of the heart rate, respiratory rate, blood pressure, body temperature and arterial oxygen saturation of the patient.

20. A method of monitoring patient status according to any claim 12, wherein the second risk estimate is determined by novelty detection by comparing the vital signs measurements to a multivariate, multimodal model of normality and obtaining a probability that the current vital signs are normal.

21. A method of monitoring patient status according to claim 12 wherein the second risk estimate is updated and a new weighted combination formed every time a new vital signs measurement is received.

22. A method of monitoring patient status according to claim 12 further comprising the step of displaying a plurality of factors most significantly influencing the combined risk estimate.

23. A computer program comprising program code means for controlling a computer to execute the method of claim 12.
